(19) United States
(12) Patent Application Publication
(10) Pub. No.: US 2022/0097056 A1
(43) Pub. Date: Mar. 31, 2022

(54) HEATING STRUCTURE, DETECTION CHIP, AND NUCLEIC ACID DETECTION DEVICE

(71) Applicant: iCare Diagnostics International Co. Ltd., New Taipei City (TW)

(72) Inventors: CHIH-HUNG WANG, New Taipei (TW); FU-KUO HUANG, New Taipei (TW); TUNG-YU WU, New Taipei (TW); YU-HSU LIN, Santa Clara, CA (US)

(21) Appl. No.: 17/488,597

(22) Filed: Sep. 29, 2021

Related U.S. Application Data
(60) Provisional application No. 63/085,368, filed on Sep. 30, 2020.

(30) Foreign Application Priority Data
May 31, 2021 (CN) 202110604898.3

(51) Int. Cl. B01L 3/00; G01N 27/447; B01L 7/00

(52) U.S. Cl. CPC B01L 3/502715; B01L 3/502707; G01N 27/44743; G01N 27/44726; B01L 7/52; C12Q 1/6806; B01L 2300/0819; B01L 2300/12; B01L 2200/16; B01L 2400/0421; B01L 2300/18

(57) ABSTRACT

A heating structure includes a substrate, a heating layer, a heat conducting layer, and a heat sensing layer. The heating layer includes at least one heating area. The heat conducting layer corresponds to the heating area. The heat sensing layer is disposed on the at least one heating area and electrically connected to the heating layer. The heating layer is used to heat the heat conducting layer. The heat sensing layer is used to sense a temperature of the heating area. A detection chip with the heating structure, and a nucleic acid detection device with the nucleic acid detection chip are also disclosed. The heating structure can make the heating temperature of the heating area more uniform and stable. The heating area of the heating structure has a lower heat loss and a higher heating efficiency.

### FIELD

The subject matter relates to nucleic detection device, and more particularly, to a heating structure, a detection chip with the heating structure, and a nucleic acid detection device with the nucleic acid detection chip.

### BACKGROUND

Molecular diagnosis, morphological detection, and immunological detection are mostly carried out in a microfluidic chip. The microfluidic chip includes a channel for carrying a detection solution. The detection solution performs a nucleic acid amplification reaction in the channel. The detection solution usually needs to be heated during the nucleic acid amplification reaction. However, the heating of the microfluidic detection chip may be uneven, resulting in a low accuracy of temperature control. Therefore, there is room for improvement in the art.

### BRIEF DESCRIPTION OF THE DRAWINGS

Implementations of the present technology will now be described, by way of example only, with reference to the attached figures.

FIG. 1 is a diagrammatic view of an embodiment of a heating structure according to the present disclosure.

FIG. 2 is a cross-sectional view of an embodiment of a heating structure according to the present disclosure.

FIG. 3 is a diagrammatic view of an embodiment of a heating layer of a heating structure according to the present disclosure.

FIG. 4 is a diagrammatic view of an embodiment of a heat conducting layer of a heating structure according to the present disclosure.

FIG. 5 is a cross-sectional view of an embodiment of a detection chip according to the present disclosure.

FIG. 6 is a diagrammatic view of an embodiment of a detection chip according to the present disclosure.

FIG. 7 is a diagrammatic view of an embodiment of a detection path in the detection chip according to the present disclosure.

FIG. 8 and FIG. 9 are photographs showing temperature changes in a detection chip according to the present disclosure when different heating zones are opened.

FIG. 10 is a diagram showing temperature changes in a detection chip according to the present disclosure when used in salt water.

FIG. 11 is a diagrammatic view of an embodiment of a nucleic acid detection kit according to the present disclosure.

FIG. 12 is a diagrammatic view of an embodiment of a nucleic acid detection device according to the present disclosure.

### DETAILED DESCRIPTION

It will be appreciated that for simplicity and clarity of illustration, where appropriate, reference numerals have been repeated among the different figures to indicate corresponding or analogous components. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to better illustrate details and features of the present disclosure.

The term “comprising,” when utilized, means “including, but not necessarily limited to”; it specifically indicates open-ended inclusion or membership in the so-described combination, group, series, and the like.

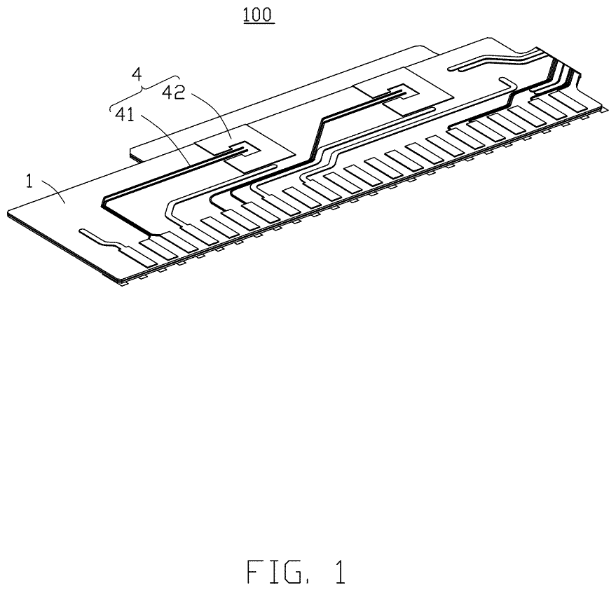

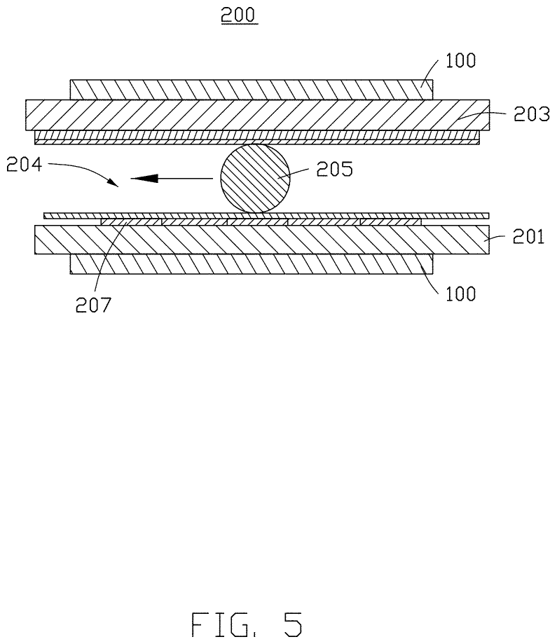

FIGS. 1 to 3 illustrate a heating structure 100, which includes a substrate 1, a heating layer 2, a heat conducting layer 3, and a heat sensing layer 4. The heating layer 2 is disposed on the substrate 1, which includes at least one heating area 21. The heat conducting layer 3 is disposed on a surface of the substrate 1 away from the heating layer 2. The heat conducting layer 3 corresponds to the heating area 21. The heat sensing layer 4 is disposed on the heating area 21 and electrically connected to the heating layer 2. The heating layer 2 is used to heat the heat conducting layer 3. The heat sensing layer 4 is used to sense a temperature of the heating area 21. Referring to FIG. 5, the heating structure 100 can be applied to a detection chip 200 for nucleic acid amplification reaction. A detection solution with nucleic acid samples is contained in the detection chip 200. The heating structure 100 is used to heat the detection solution to initiate the nucleic acid amplification reaction.

In an embodiment, the substrate 1 is made of an insulating resin selected from a group consisting of epoxy resin, polyphenylene oxide (PPO), polyimide (PI), polyethylene terephthalate (PET), and polyethylene naphthalate (PEN), and any combination thereof.

In an embodiment, the substrate 1 is made of PI or PET, which can reduce a cost of the heating structure 100 and the detection chip 200.

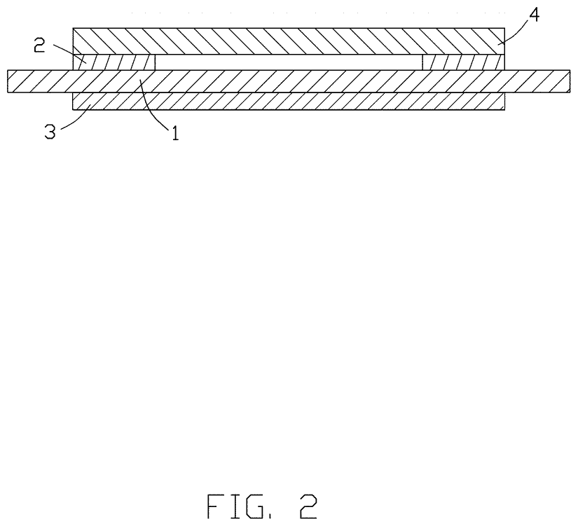

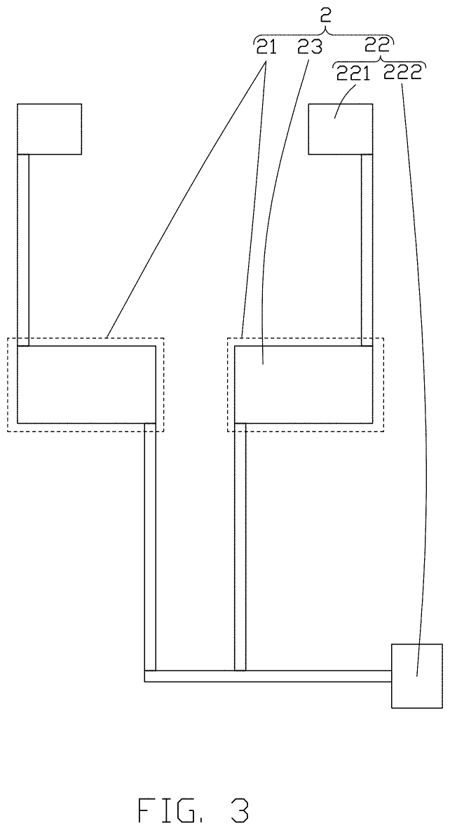

Referring to FIGS. 2 and 3, the heating layer 2 further includes a heating circuit 22 and a heating resistance 23 disposed on the substrate 1. The heating circuit 22 includes the at least one heating area 21. Each of the heating areas 21 includes one heating resistance 23 therein. The number of the heating area(s) 21 can be set according to actual needs. When the heating circuit 22 is energized, the heating resistance 23 in the heating area 21 is energized and generate heat.

In an embodiment, the heating circuit 22 is provided with a power electrode 221 and a grounding electrode 222 corresponding to each heating area 21. The power electrode 221 and the grounding electrode 222 corresponding to each heating area 21 are respectively disposed on opposite sides of the heating resistance 23 in the heating area 21, which is conducive to heat the whole heating area 21 uniformly.

In an embodiment, a plurality of heating areas 21 are disposed on the heating layer 2. Two adjacent heating areas 21 are spaced apart from each other. Each heating area 21 includes one heat conducting layer 3 therein. The heating areas 21 can be heated independently. A temperature of each of the heating areas 21 is different from each other, thereby allowing the nucleic acid amplification reaction to perform at different temperatures. A certain distance is between two adjacent heating areas 21, which can reduce a temperature interference between the two heating areas 21 and facilitate the accurate temperature control of each heating area 21.

In an embodiment, the heating circuit 22 can be formed on the substrate 1 by plane printing or 3D printing. The heating circuit 22 can also be formed by exposure and development process.

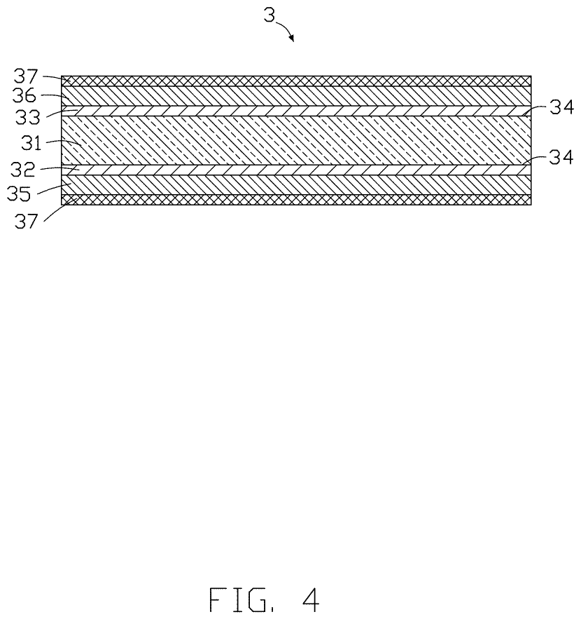

Referring to FIG. 4, the heat conducting layer 3 includes a metal layer 31, a first graphite layer 32, and a second graphite layer 33. The first graphite layer 32 and the second graphite layer 33 are disposed on two opposite surfaces of the metal layer 31. The first graphite layer 32 faces the heating layer 2, that is, the first graphite layer 32 is disposed on a surface of the substrate 1 away from the heating layer 2. The second graphite layer 33 faces a device to be heated (not shown). The heat conducting layer 3 can make the heating of the heating area 21 to be uniform due to a uniformity heat conduction in a horizontal direction of the graphite layer. At the same time, the heat conducting layer 3 can avoid violent temperature change during heating the heating area 21.

In an embodiment, a first heat conducting adhesive layer 35 is disposed between the first graphite layer 32 and the substrate 1. A second heat conducting adhesive layer 36 is disposed on a surface of the second graphite layer 33 away from the substrate 1. The heat conducting layer 3 is connected to the surface of the substrate 1 away from the heating layer 2 through the first heat conducting adhesive layer 35. The heat conducting layer 3 is further connected to a surface of the device to be heated through the second heat conducting adhesive layer 36.

In an embodiment, a thickness of each of the first heat conducting adhesive layer 35 and the second heat conducting adhesive layer 36 is about 0.1 mm.

In an embodiment, the first thermal conducting adhesive layer 35 or the second thermal conducting adhesive layer 36 may be made of, but is not limited to a thermal conductive double-sided adhesive.

In an embodiment, the first thermal conducting adhesive layer 35 may be made of, but is not limited to an acrylic adhesive. The second thermal conducting adhesive layer 36 may be made of, but is not limited to a silicone adhesive.

In an embodiment, a thickness of the metal layer 31 is in a range from 0.05 mm to 0.15 mm.

In an embodiment, the metal layer 31 may be, but is not limited to a copper foil.

In an embodiment, a thickness of each of the first graphite layer 32 and the second graphite layer 33 is in a range from 0.02 mm to 0.03 mm. Due to an excellent thermal conductivity of graphite in the horizontal direction of the first graphite layer 32 and the second graphite layer 33, a thermal conductivity can be more uniform, a heat loss can be lower, and a heating efficiency can be higher. By disposing the first graphite layer 32 and the second graphite layer 33 on both surfaces of the metal layer 31, the heat can be evenly stored, avoiding a violent temperature change during heating the heating area 21. Thus, the heat can be uniformly distributed over the heating area 21. The heat loss is lower, the heating efficiency is higher, and the temperature control is more accurate.

In an embodiment, two third heat conducting adhesive layers 34 are disposed between the metal layer 31 and the first graphite layer 32 and between the metal layer 31 and the second graphite layer 33. The first graphite layer 32 and the second graphite layer 33 are bonded on two surfaces of the metal layer 31 through the two third heat conducting adhesive layers 34 to form a composite heat conductive layer structure. The method for forming the heat conducting layer 3 is simple. The thickness of the heat conducting layer 3 is uniform to ensure uniform heating. The heat conducting layer 3 can be shaped according to the surface areas of the heating areas 21.

In an embodiment, a thickness of each of the third heat conducting adhesive layers 34 is in a range from 0.01 mm to 0.03 mm.

In an embodiment, before the heat conducting layer 3 is pasted on the heating area 21, two release layers 37 are disposed on a surface of the first heat conducting adhesive layer 35 away from the metal layer 31 and a surface of the second heat conducting adhesive layer 36 away from the metal layer 31.

Referring to FIGS. 1 and 3, the heat sensing layer 4 includes a temperature sensing circuit 41 and a temperature sensor 42 disposed on the heating area 21. The temperature of the heating area 21 can be sensed through the temperature sensor 42.

In an embodiment, a surface area of the temperature sensor 42 is roughly equal to a surface area of the heating area 21. When the temperature sensor 42 is connected to a surface of the heating area 21 away from the heat conducting layer 3, a temperature change in all parts of the heating area 21 can be sensed, ensuring an accuracy and stability of temperature control in all parts of the heating area 21.

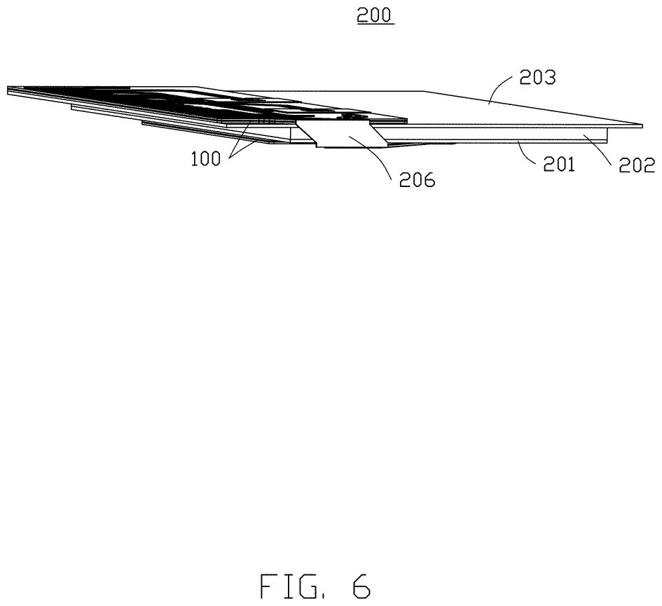

FIGS. 5 to 6 illustrate a detection chip 200, which includes a first cover plate 201, a second cover plate 203, a spacer layer 202, and the heating structure 100. Two opposite surfaces of the spacer layer 202 are in contact with the first cover plate 201 and the second cover plate 203. The first cover plate 201, the spacer layer 202, and the second cover plate 203 cooperatively define a channel 204 for carrying a detection solution 205. The heating structure 100 is disposed on a surface of the first cover plate 201 away from the channel 204 and/or the second cover plate 203 away from the channel 204. The heating structure 100 is used to heat the detection solution 205 to initiate the nucleic acid amplification reaction.

In an embodiment, referring to FIGS. 5 and 6, two heating structures 100 are disposed on a surface of the first cover plate 201 away from the channel 204 and a surface of the second cover plate 203 away from the channel 204. The two heating structures 100 are electrically connected to each other through a connecting part 206. The two heating structures 100 and the connecting part 206 are an integrated structure. The two heating structures 100 can heat the detection solution 205 in the channel 204 more evenly. In addition, the electrical connection of the two heating structures 100 is realized through the connecting part 206. The connecting heating structures 100 and the connecting part 206 as an integrated structure result in a convenient assembly of the heating structure 100 in the detection chip 200. Furthermore, output wirings are only designed on one of the two heating structures 100, which is convenient to connect to a power supply.

In an embodiment, the heating structures 100 can be bonded on the surface of the first cover plate 201 and/or the surface of the second cover plate 203 through the second heat conducting adhesive layer 36.

In an embodiment, the second heat conducting adhesive layer 36 is made of a silicone adhesive. The first cover plate 201 and the second cover plate 203 can be glass cover plates. The silicone adhesive has excellent properties such as high temperature resistance and weather resistance, which can stably bond the heating structure 100 on the glass cover plates.

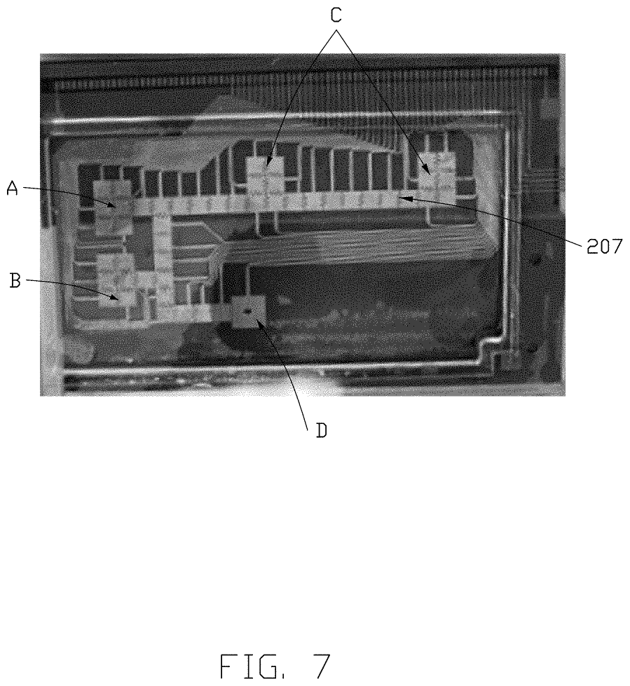

Referring to FIGS. 5 and 7, the channel 204 includes a detection path 207. The detection solution 205 can flow in the detection path 207. The detection path 207 can be divided into a plurality of areas according to different purposes, including a sample adding area “A”, a reagent storage area “B”, a plurality of nucleic acid amplification areas “C”, and a solution outlet area “D”. The detection solution 205 is added in the sampling area “A” through a sampling port. The reagent storage area “B” is used to store fluorescent reagents (such as fluorescent dyes or fluorescent probes). The detection solution 205 performs the nucleic acid amplification reaction in the nucleic acid amplification areas “C”. A number of the nucleic acid amplification areas “C” can be set according to an actual detection requirement.

After the detection solution 205 enters the sampling area “A”, the detection solution 205 moves to the nucleic acid amplification areas “C” and performs the nucleic acid amplification reaction to form an amplified product. When the nucleic acid amplification reaction is completed, the amplified product is moved to the reagent storage area “B” and mixed with the fluorescent reagent to obtain a mixture. The mixture then enters the next step (such as electrophoretic detection).

In an embodiment, the number of nucleic acid amplification regions “C” is two. Each of the two nucleic acid amplification regions “C” corresponds to one heating area 21. The heating structure 100 includes two heating areas 21 and two heat conducting layers 3. The heating temperatures of the two nucleic acid amplification regions “C” are different, so that different stages of nucleic acid amplification reaction of the detection solution 205 can be performed at different temperatures.

In an embodiment, the two heating temperatures of the two nucleic acid amplification regions “C” are in ranges from 90° C. to 105° C. and from 40° C. to 75° C. respectively.

In yet other embodiment, the number of the nucleic acid amplification regions “C” may be three or more according to different stages of the nucleic acid amplification reaction. The three heating temperatures of the three nucleic acid amplification areas “C” are in ranges from 90° C. to 105° C., from 68° C. to 75° C., and from 40° C. to 65° C.

In yet another embodiment, the reagent storage area “B” is also heated by the heating structure 100. The mixer includes the amplified product, and the fluorescent reagent is preheated in the reagent storage area “B”.

Referring to FIGS. 3, 7, 8 and 9, the detection path 207 includes three heating areas 21. The heating temperatures of the three heating areas 21 are in ranges from 90° C. to 105° C., from 68° C. to 75° C., and from 40° C. to 65° C. A certain distance is between any two adjacent heating areas 21. During the heating process, the three heating areas 21 can be heated at the same time, or anyone of the three heating areas 21 can be heated first. In an embodiment, since the detection solution 205 stays in the heating area 21 with the temperature ranges from 90° C. to 105° C. for a longer time, such heating area 21 can be heated first. Then, the other heating areas 21 can be heated.

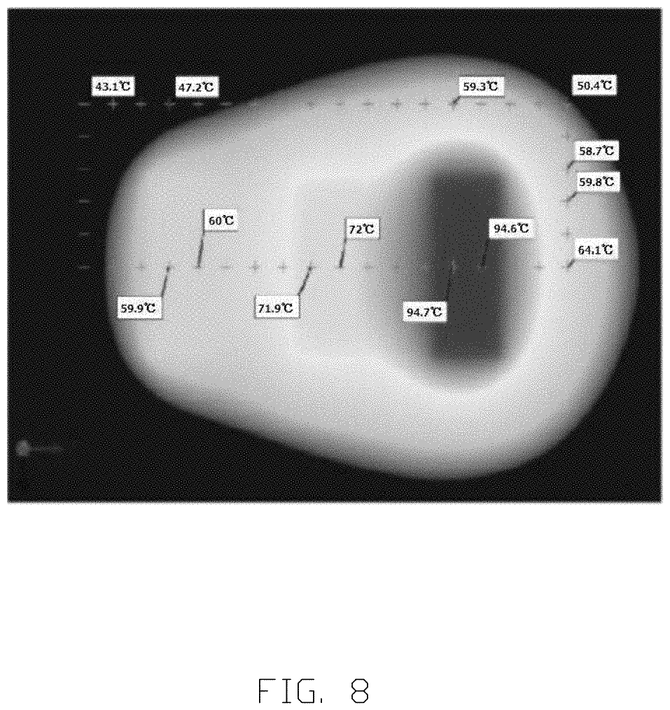

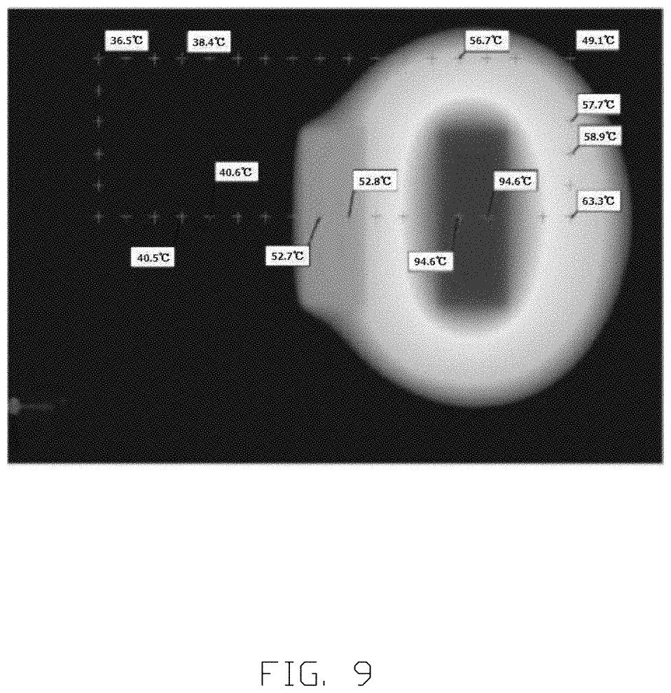

Referring to FIGS. 8 and 9, two photographs showing temperature changes when different numbers of the heating areas 21 are heated under an ambient temperature of 30° C. When the three heating areas 21 are heated to 95° C., 72° C. and 60° C. (as shown in FIG. 8), the temperature of the first heating area 21 remains at 95° C. When the first heating area 21 is heated to 95° C. (as shown in FIG. 9), the temperature of the first heating area 21 also keeps remains at 95° C. Thus, when at least two of the heating areas 21 are heated at the same time, the temperature interference between two adjacent heating areas 21 can be ignored. Therefore, the heating structure 100 can accurately control the heating temperature of different heating areas 21.

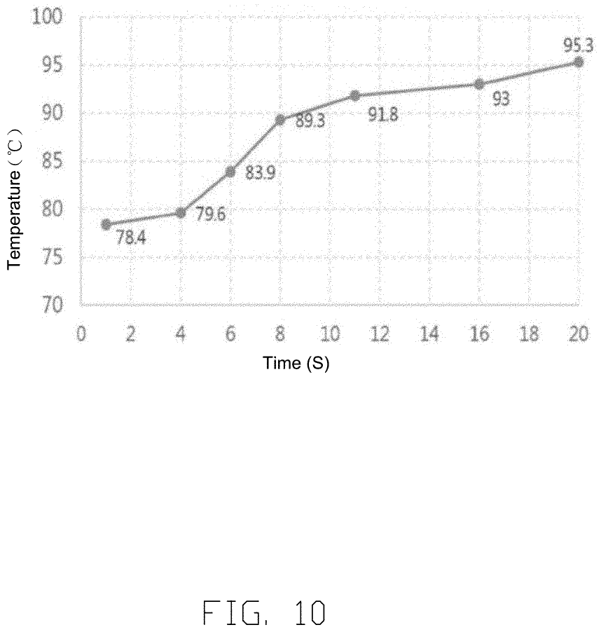

We heat the salt water in the detection chip 200, and a diagram showing temperature changes in a detection chip 200 is obtained. Referring to FIG. 10, the temperature of the salt water rises quickly over time without much fluctuation, which indicates that the heating structure 100 has a lower heat loss and a higher heating efficiency, and the temperature control is more accurate.

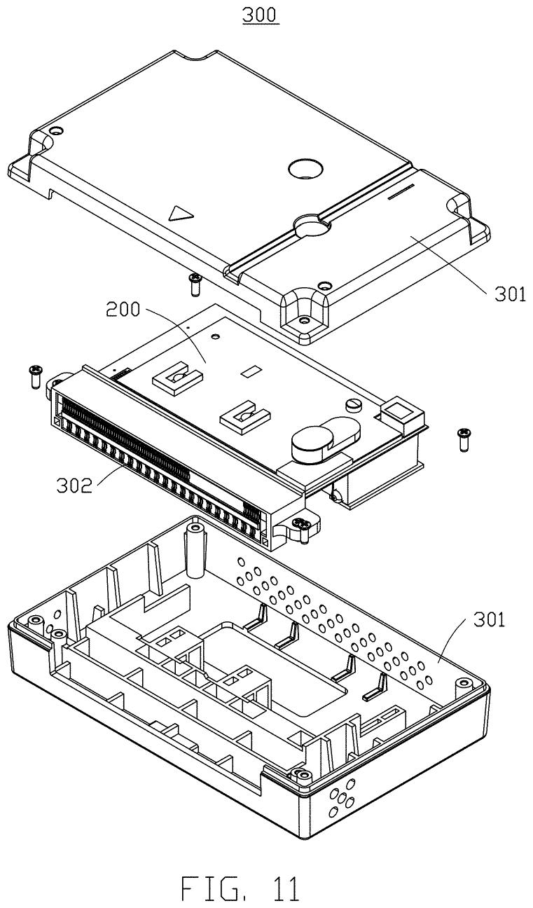

FIG. 11 illustrates a nucleic acid detection kit 300, which includes a kit body 301, a detection chip 200, and a connector 302. The detection chip 200 is disposed in the kit body 301. The detection chip 200 is electrically connected to the connector 302.

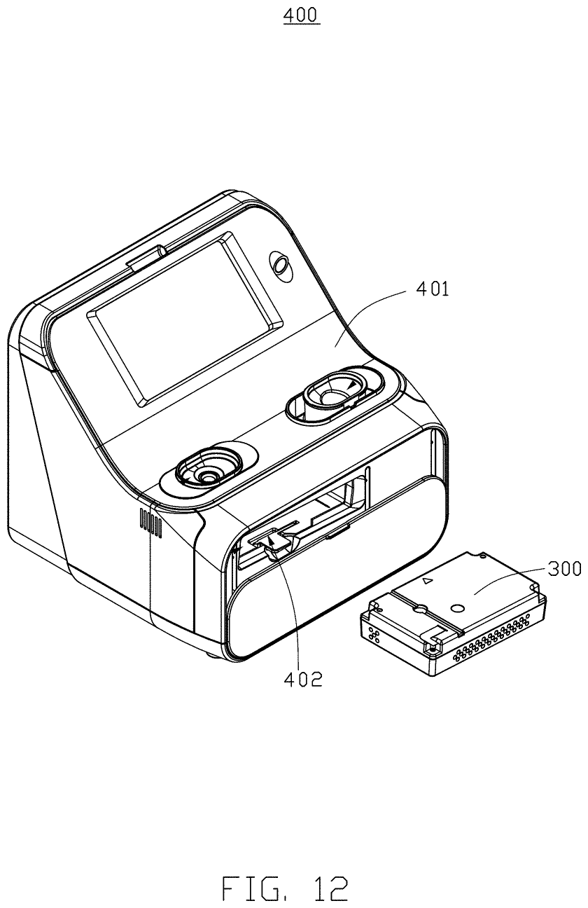

FIG. 12 illustrates a nucleic acid detection device 400, which includes a host 401 and the nucleic acid detection kit 300. The host 401 includes a mounting groove 402. The nucleic acid detection kit 300 is detachably disposed in the mounting groove 402.

With the above configuration, the heating structure 100 can uniformly heat the heating area 21 by adding the heat conducting layer 3 between the heating layer 2 and the heat sensing layer 4. The temperature of the heating area 21 can be accurately sensed through the heat sensing layer 4, which is convenient for the temperature control of the heating area 21. The heat conducting layer 3 can make the heating of the heating area 21 to be uniform due to a uniformity heat conduction in a horizontal direction of the graphite layer. At the same time, the heat conducting layer 3 can avoid a violent temperature change during the heating process of the heating area 21. The heat conducting layer 3 also allows the heating area 21 to have a lower heat loss and a higher heating efficiency.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure, up to and including, the full extent established by the broad general meaning of the terms used in the claims.

What is claimed is:

1. A heating structure, comprising:
a substrate;
a heating layer;
a heat conducting layer; and
a heat sensing layer;
wherein the heating layer is disposed on the substrate, the heating layer comprises at least one heating area, the heat conducting layer is disposed on a surface of the substrate away from the heating layer, the heat conducting layer corresponds to the at least one heating area, the heat sensing layer is disposed on the at least one heating area and electrically connected to the heating layer, the heating layer is configured to heat the heat conducting layer, the heat sensing layer is configured to sense a temperature of the at least one heating area.

2. The heating structure of claim 1, further comprising a first heat conducting adhesive layer and a second heat conducting adhesive layer, wherein the first heat conducting adhesive layer is disposed between the heat conducting layer and the substrate, the second heat conducting adhesive layer is disposed on a surface of the heat conducting layer away from the substrate.

3. The heating structure of claim 1, wherein the heat conducting layer comprises a metal layer, a first graphite layer, and a second graphite layer, the first graphite layer and the second graphite layer are disposed on two opposite surfaces of the metal layer, the first graphite layer is disposed on a surface of the substrate away from the heating layer.

4. The heating structure of claim 3, wherein a thickness of the metal layer is in a range from 0.05 mm to 0.15 mm; and
a thickness of each of the first graphite layer and the second graphite layer is in a range from 0.02 mm to 0.03 mm.

5. The heating structure of claim 3, wherein two third heat conducting adhesive layers are disposed between the metal layer and the first graphite layer and between the metal layer and the second graphite layer.

6. The heating structure of claim 5, wherein a thickness of each of the third heat conducting adhesive layers is in a range from 0.01 mm to 0.03 mm.

7. The heating structure of claim 1, wherein a plurality of heating areas is disposed on the heating layer, two adjacent of the plurality of adjacent heating areas are spaced apart from each other, the heat conducting layer is disposed on each of the plurality of heating areas.

8. A detection chip, comprising:
a heating structure, comprising:
a substrate;
a heating layer;
a heat conducting layer; and
a heat sensing layer;
wherein the heating layer is disposed on the substrate, the heating layer comprises at least one heating area, the heat conducting layer is disposed on a surface of the substrate away from the heating layer, the heat conducting layer corresponds to the at least one heating area, the heat sensing layer is disposed on the at least one heating area and electrically connected to the heating layer, the heating layer is configured to heat the heat conducting layer, the heat sensing layer is configured to sense a temperature of the at least one heating area;
a first cover plate;
a second cover plate; and
a spacer layer;
wherein two opposite surfaces of the spacer layer are in contact with the first cover plate and the second cover plate, the first cover plate, the spacer layer, and the second cover plate cooperatively define a channel for carrying a detection solution, the heating structure is disposed on a surface of the first cover plate away from the channel and/or the second cover plate away from the channel, the heating structure is configured to heat the detection solution.

9. The detection chip of claim 8, wherein the heating structure further comprises a first heat conducting adhesive layer and a second heat conducting adhesive layer, the first heat conducting adhesive layer is disposed between the heat conducting layer and the substrate, the second heat conducting adhesive layer is disposed on a surface of the heat conducting layer away from the substrate.

10. The detection chip of claim 8, wherein the heat conducting layer comprises a metal layer, a first graphite layer, and a second graphite layer, the first graphite layer and the second graphite layer are disposed on two opposite surfaces of the metal layer, the first graphite layer is disposed on a surface of the substrate away from the heating layer.

11. The detection chip of claim 10, wherein a thickness of the metal layer is in a range from 0.05 mm to 0.15 mm; and
a thickness of each of the first graphite layer and the second graphite layer is in a range from 0.02 mm to 0.03 mm.

12. The detection chip of claim 10, wherein two third heat conducting adhesive layers are disposed between the metal layer and the first graphite layer and between the metal layer and the second graphite layer.

13. The detection chip of claim 12, wherein a thickness of each of the third heat conducting adhesive layers is in a range from 0.01 mm to 0.03 mm.

14. The detection chip of claim 8, wherein a plurality of heating areas is disposed on the heating layer, two adjacent of the plurality of adjacent heating areas are spaced apart from each other, the heat conducting layer is disposed on each of the plurality of heating areas.

15. The detection chip of claim 8, wherein two heating structures are disposed on a surface of the first cover plate away from the channel and a surface of the second cover plate away from the channel, the two heating structures are electrically connected through a connecting part, and the two heating structures and the connecting part are an integrated structure.

16. A nucleic acid detection device, comprising:
a nucleic acid detection kit, comprising:
a detection chip, comprising:
a heating structure, comprising:
a substrate;
a heating layer;
a heat conducting layer; and
a heat sensing layer;
wherein the heating layer is disposed on the substrate, the heating layer comprises at least one heating area, the heat conducting layer is disposed on a surface of the substrate away from the heating layer, the heat conducting layer corresponds to the at least one heating area, the heat sensing layer is disposed on the at least one heating area and electrically connected to the heating layer, the heating layer is configured to heat the heat conducting layer, the heat sensing layer is configured to sense a temperature of the at least one heating area;
a first cover plate;
a second cover plate; and
a spacer layer;
wherein two opposite surfaces of the spacer layer are in contact with the first cover plate and the second cover plate, the first cover plate, the spacer layer, and the second cover plate cooperatively define a channel for carrying a detection solution, the heating structure is disposed on a surface of the first cover plate away from the channel and/or the second cover plate away from the channel, the heating structure is configured to heat the detection solution;
a kit body;
a connector; and
wherein the detection chip is disposed in the kit body and electrically connected to the connector; and
a host;
wherein the host comprises a mounting groove, the nucleic acid detection kit is detachably disposed in the mounting groove.

17. The nucleic acid detection device of claim 16, wherein the heating structure further comprises a first heat conducting adhesive layer and a second heat conducting adhesive layer, the first heat conducting adhesive layer is disposed between the heat conducting layer and the substrate, the second heat conducting adhesive layer is disposed on a surface of the heat conducting layer away from the substrate.

18. The nucleic acid detection device of claim 16, wherein the heat conducting layer comprises a metal layer, a first graphite layer, and a second graphite layer, the first graphite layer and the second graphite layer are disposed on two opposite surfaces of the metal layer, the first graphite layer is disposed on a surface of the substrate away from the heating layer.

19. The nucleic acid detection device of claim 18, wherein two third heat conducting adhesive layers are disposed between the metal layer and the first graphite layer and between the metal layer and the second graphite layer respectively.

20. The nucleic acid detection device of claim 16, wherein a plurality of heating areas is disposed on the heating layer, two adjacent of the plurality of adjacent heating areas are spaced apart from each other, the heat conducting layer is disposed on each of the plurality of heating areas.
